(19) United States
(12) Patent Application Publication
(10) Pub. No.: US 2019/0192036 A1
(43) Pub. Date: Jun. 27, 2019

(54) ELECTRONIC DEVICE AND METHOD OF MANUFACTURING ELECTRONIC DEVICE

(71) Applicant: FUJITSU LIMITED, Kawasaki-shi (JP)

(72) Inventors: Taiji Sakai, Yokohama (JP); Hideki Kitada, Atsugi (JP); KOZO SHIMIZU, Atsugi (JP)

(73) Assignee: FUJITSU LIMITED, Kawasaki-shi (JP)

(21) Appl. No.: 16/291,024

(22) Filed: Mar. 4, 2019

Related U.S. Application Data
(63) Continuation of application No. PCT/JP2016/077488, filed on Sep. 16, 2016.

(51) Int. Cl. A61B 5/0408; A61B 10/00; A61B 5/145; C09J 9/02

(52) U.S. Cl. CPC A61B 5/04087; A61B 10/0064; C08J 2300/10; C09J 9/02; A61B 5/14517

(57) ABSTRACT

An electronic device includes: a polymer film that is to melt at a predetermined temperature higher than a body temperature; at least one electronic component provided in the polymer film; and a first hydrophobic film provided on an opposite surface of the polymer film to a side of the polymer film to be attached to skin.

### CROSS-REFERENCE TO RELATED APPLICATION

This application is a continuation application of International Application PCT/JP2016/077488 filed on Sep. 16, 2016 and designated the U.S., the entire contents of which are incorporated herein by reference.

### FIELD

The embodiments discussed herein relate to an electronic device and a method of manufacturing the electronic device.

### BACKGROUND

Nowadays, there are proposed wearable devices attached to the skin to detect information such as biological signals or a secretion such as sweat produced by a biological body.

Related art is disclosed in Japanese Laid-open Patent Publication No. 2014-237060, Japanese National Publication of International Patent Application No. 2015-513104 and Japanese Laid-open Patent Publication No. 2016-125023.

### SUMMARY

According to an aspect of the embodiments, an electronic device includes: a polymer film that is to melt at a predetermined temperature higher than a body temperature; at least one electronic component provided in the polymer film; and a first hydrophobic film provided on an opposite surface of the polymer film to a side of the polymer film to be attached to skin.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 illustrates an embodiment of an electronic device;

FIGS. 2A to 2F illustrate an example of a method of manufacturing the electronic device illustrated in FIG. 1;

FIGS. 3A to 3C illustrate an example of a procedure for removing the electronic device illustrated in FIG. 1 from the skin;

FIG. 4 illustrates another embodiment of the electronic device;

FIGS. 5A to 5F illustrate an example of a method of manufacturing the electronic device illustrated in FIG. 4;

FIGS. 6A to 6E illustrate another example of the method of manufacturing the electronic device illustrated in FIG. 4;

FIGS. 7A to 7C illustrate yet another example of the method of manufacturing the electronic device illustrated in FIG. 4;

FIG. 8 illustrates another embodiment of the electronic device;

FIG. 9 illustrates another embodiment of the electronic device;

FIG. 10 illustrates another embodiment of the electronic device;

FIGS. 11A to 11E illustrate an example of the method of manufacturing the electronic device illustrated in FIG. 10;

FIGS. 12A to 12C illustrate steps of the method of manufacturing the electronic device following the method illustrated in FIGS. 11A to 11E;

FIG. 13 illustrates another embodiment of the electronic device;

FIG. 14 illustrates another embodiment of the electronic device;

FIGS. 15A to 15E illustrate an example of the method of manufacturing the electronic device illustrated in FIG. 14; and

FIG. 16 illustrates another embodiment of the electronic device.

### DESCRIPTION OF EMBODIMENTS

A device that detects biological signals includes a hydrophilic conductive polymer film to be attached to the skin, a polymer gel film, and a protective sheet covering the polymer gel film. A device that detects components of sweat includes a plurality of gates, bridges, and pads. The time required for the sweat to be transmitted through the gates varies from one gate to another. The bridges gradually dissolve due to sweat transmitted from the gates. The pads collect sweat transmitted from the bridges. There is also proposed a technique for attaching a wearable device to the skin with an adhesive. An organic material having crystallinity is added this adhesive so as to reduce the adhesion of the adhesive when heated.

In use, the above-described wearable device is attached to the skin such that the wearable device is in close contact with the skin. After use of the device, the wearable device in close contact with the skin is removed from the skin. This may damage the skin when the device is removed from the skin. Even when the adhesive the adhesion of which reduces due to heating is used, there still remains adhesion. Thus, the skin may be damaged when the device is removed from the skin. When the adhesive is dissolved using a solvent or the like so as to remove the device from the skin, the skin may be damaged by the solvent.

In one aspect, damage to the skin when an electronic device attached to the skin is removed from the skin may be surpressed.

Hereinafter, embodiments will be described with reference to the drawings.

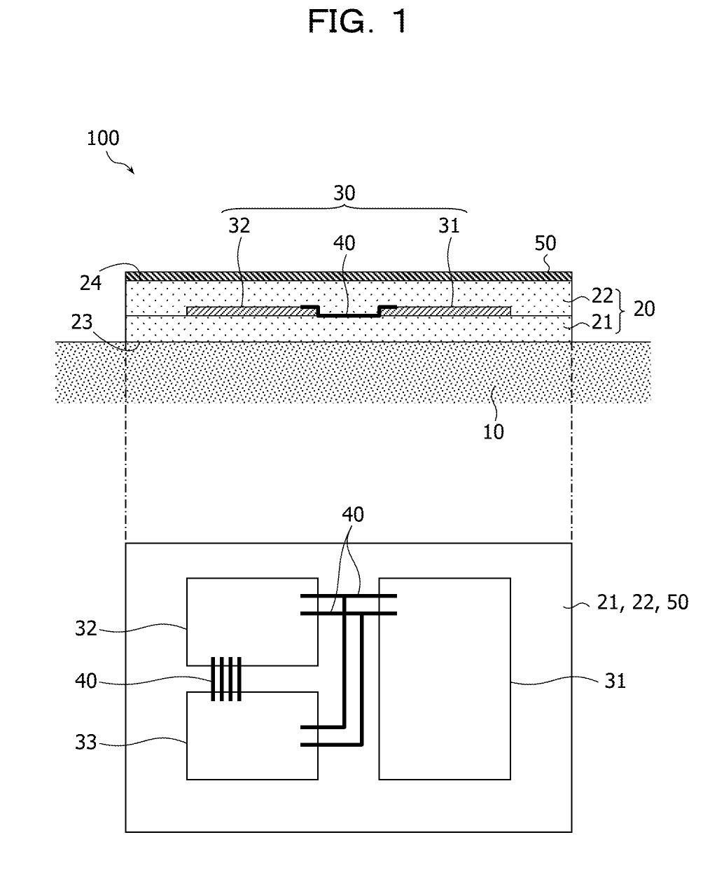

FIG. 1 illustrates an embodiment of an electronic device. An electronic device 100 illustrated in FIG. 1 is to be attached to skin 10 of, for example, a human body and collects biological information such as body temperature or heart rate, for example. An upper portion of FIG. 1 illustrates a section of the electronic device 100, and a lower portion of FIG. 1 illustrates an upper surface of the electronic device 100.

The electronic device 100 includes a water-soluble polymer film 20 having an inner surface 23 which is a surface to be attached to the skin 10, a predetermined number of electronic components 30 (31, 32, 33) disposed in the polymer film 20, and wiring 40 that couples the terminals of the electronic components 30 to one another. The electronic device 100 is to be attached to the skin 10 by utilizing an adhesive property of the polymer film 20. In addition, the electronic device 100 has a hydrophobic film 50 provided on an outer surface 24 which is a surface opposite to the inner surface 23 of the polymer film 20. The hydrophobic film 50 is an example of a first hydrophobic film. The hydrophobic film 50 is formed of, for example, a powdered silicone resin or a powdered fluororesin and has a waterproof function with which water or the like is repelled. By the hydrophobic film 50, even when water or the like is applied to the electronic device 100 attached to the skin 10, dissolution of the water-soluble polymer film 20 is able to be suppressed. As a result, failure of the electronic device 100 due to dissolution of the polymer film 20 may be suppressed. When a single electronic component 30 is mounted on the electronic device 100, the wiring 40 is not necessarily formed.

The polymer film 20 includes laminated polymer films 21 and 22, and the electronic components 30 are disposed at the interface between the polymer films 21 and 22. For example, the melting temperature of the polymer film 21 is higher than the melting temperature of the polymer film 22. By setting the melting temperature of the polymer film 21 to be higher than the melting temperature of the polymer film 22, reaction between the polymer film 21 and sweat produced from the skin 10 may be suppressed. For example, the polymer film 21 is formed of polyethylene glycol having a molecular weight of about 2,500, and the polymer film 22 is formed of polyethylene glycol having a molecular weight of about 2,000. By making the molecular weight of the polyethylene glycol forming the polymer film 21 larger than the molecular weight of the polyethylene glycol forming the polymer film 22, the melting temperature of the polymer film 21 becomes higher than the melting temperature of the polymer film 22. The melting temperature of the polymer films 21 and 22 is about 50 degrees Celsius, which is higher than the body temperature (the surface temperature of the skin). Therefore, in a state where the electronic device 100 is attached to the skin 10, neither of the polymer films 21 and 22 are deformed by melting, and the electronic device 100 maintains the shape illustrated in FIG. 1. Here, the melting temperature is a temperature at which the polymer film 20 begins to melt, and the polymer film 20 exceeding the melting temperature is deformed by gravity or an external force. The melting temperatures of the polymer films 21 and 22 may be set equal to each other.

The polymer film 20 may be formed of another material such as gelatin whose melting temperature is higher than the body temperature or a mixture of polyethylene glycol and gelatin. Alternatively, the polymer film 20 may be formed of a mixture of polyethylene glycol and collagen, a mixture of polyethylene glycol and starch, a mixture of gelatin and collagen, or a mixture of gelatin and starch. For example, the polymer film 20 may be formed of a mixture containing at least one of polyethylene glycol and gelatin and at least one of collagen and starch. By using naturally occurring collagen, starch or gelatin as the material of the polymer film 20, an environmental burden caused by the manufacture of the electronic device 100 may be reduced. Hereinafter, polyethylene glycol and gelatin are also referred to as meltable materials that melt by heating, and collagen and starch are also referred to as non-meltable materials that do not melt by heating.

The melting temperature of the meltable material is set to such a temperature that is higher than the body temperature and does not cause burns. Preferably the melting temperature is set in a range of, for example, 45 to 60 degrees Celsius. In order to deform the electronic device 100 attached to the skin 10 by heating so as to disintegrate the electronic device 100, it is preferable that the meltable material contained in the polymer film 20 be more than the non-meltable material contained in the polymer film 20 in, for example, volume ratio. When the meltable material contained in the polymer film 20 is more than the non-meltable material contained in the polymer film 20, the non-meltable material is distributed in the meltable material. Therefore, when the meltable material melts due to heating, the non-meltable material is separated into pieces, and the electronic device 100 is likely to be disintegrated. In contrast, when non-meltable material contained in the polymer film 20 is more than the meltable material contained in the polymer film 20, the meltable material is distributed in the non-meltable material. Therefore, even when the meltable material is melted by heating, the non-meltable material is not separated into pieces, and the electronic device 100 is less likely to be disintegrated.

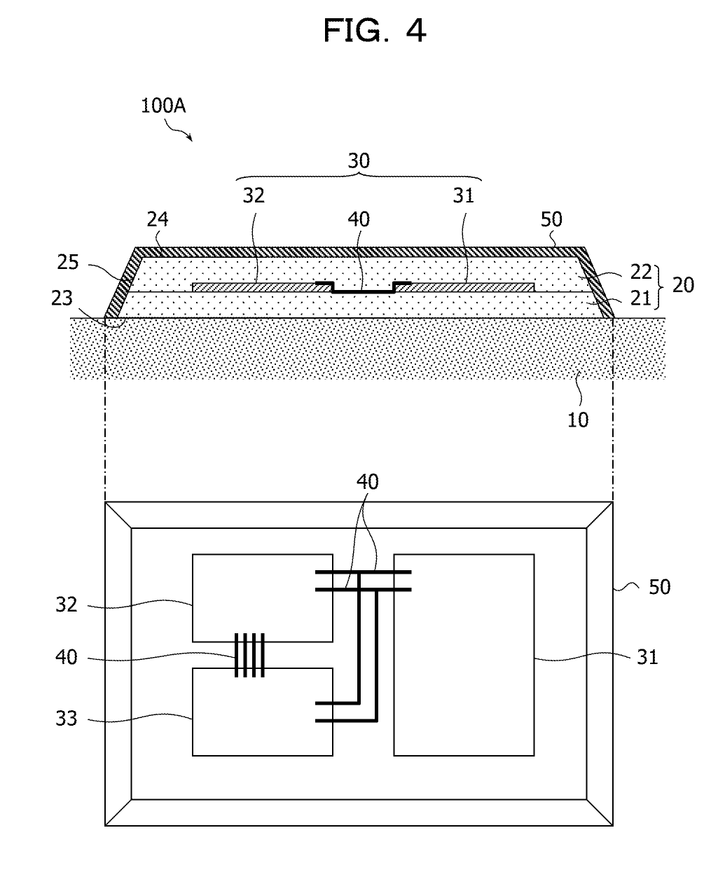

By forming the polymer film 20 from a material in which the meltable material and the non-meltable material are mixed, it is possible to increase the rigidity of the polymer film 20 compared to the case where the polymer film 20 is formed only with the meltable material. Thus, the electronic device 100 may become difficult to be deformed even when subjected to an external force or the like, and accordingly, the reliability of the wiring 40 and the like may be improved. Furthermore, even when the rigidity is high, as described with reference to FIG. 4, by heating the polymer film 20, the electronic device 100 is able to be washed off together with the electronic components 30 without being pulled off from the skin 10.

For example, the electronic component 31 is a battery, the electronic component 32 is a temperature sensor or the like, and the electronic component 33 is a communication interface such as a Bluetooth module (Bluetooth is a registered trademark). The electronic component 31 may instead be a solar panel or a battery with a solar panel. When a temperature sensor is used as the electronic component 32, body temperature of a patient or the like is measurable at a medical site or a nursing care site.

In the case of measuring the heart rate of a patient or the like, a vibration sensor or an optical module including an infrared LED (light emitting diode) for outputting infrared light and a receiving unit for infrared light is used as the electronic component 32. In the case of measuring the blood pressure of a patient or the like, a pressure sensor or a piezoelectric sensor is used as the electronic component 32. In the case of acquiring an electrocardiogram of a patient or the like, an acceleration sensor is used as the electronic component 32. In the case of detecting breathing of a patient or the like, an acceleration sensor or a piezoelectric sensor is used as the electronic component 32. In the case of detecting the state of sleep of a patient or the like, an acceleration sensor or a pressure sensor is used as the electronic component 32.

The electronic component 32 may include a plurality of sensors, or a plurality of types of electronic components 32 (sensors) may be mounted on the electronic device 100. In this manner, the electronic component 32 mounted on the electronic device 100 is selected according to the type of biological information to be collected through the skin.

The types and the number of the electronic components 30 mounted on the electronic device 100 are not limited to the above description. For example, instead of the Bluetooth module, a radio frequency identification (RFID) module may be mounted on the electronic device 100. A display component such as electronic paper or an organic electroluminescence (EL) display may be mounted on the electronic device 100. The electronic device 100 on which the display component is mounted is able to function as, for example, a tag that displays information for identifying a patient or the like. Alternatively, the electronic device 100 on which the display component is mounted is able to function as electronic decoration or an electronic tattoo that decorates or gives a design appearance to the skin 10.

Polyethylene glycol and gelatin are transparent, and the hydrophobic film 50 formed of silicone resin or fluororesin is substantially transparent in the case of a thin film. Thus, characters, images, or the like displayed on the display of the display component are visible from the surface of the electronic device 100. In the case where the polymer film 22 contains starch or collagen, the thickness of the polymer film 22 is desirably reduced as much as possible in order to improve the visibility of the display. In addition, by making the thickness of the display component larger than the other electronic components mounted on the electronic device 100, the thickness of the polymer film 22 on the display component is made smaller than the thickness of the polymer film 22 on the other electronic components. This may improve the visibility of the display compared to the case of using a display component having the same thickness as other electronic components. Alternatively, instead of forming the polymer film 22 and the hydrophobic film 50 on the display, the display may be exposed at the surface of the electronic device 100.

The wiring 40 connecting the terminals of the electronic component 31 to the terminals of the electronic components 32, 33 is power lines, and the wiring 40 connecting the terminals of the electronic components 32, 33 to one another is signal lines. The electronic component 32 detects biological information such as body temperature through the skin 10 at a predetermined frequency and outputs the detected biological information to the electronic component 33. The electronic component 33 transmits the received biological information to an external computer device (not illustrated). Then, the biological information detected by the electronic device 100 is accumulated in the computer device. The electronic component 32 or the electronic component 33 may include a storage unit that stores the biological information. In this case, the electronic device 100 may transmit the biological information held therein to the computer device in accordance with a request from the computer device.

The thickness of the polymer film 21 is preferably as small as possible in order to suppress degradation of sensitivity for detecting the biological information by the electronic component 32. The polymer film 22 is preferably formed to have such a thickness that the polymer film 22 is able to protect the electronic components 31, 32, 33. For example, the thickness of the polymer film 21 is about 0.5 to 2 mm, and the thickness of the polymer film 22 is about 2 to 3 mm. For example, the thickness of the hydrophobic film 50 is about 5 to 50 microns. In FIG. 1 and other drawings, the thickness and the aspect ratio of each element are different from the thickness and the aspect ratio of the actually formed element.

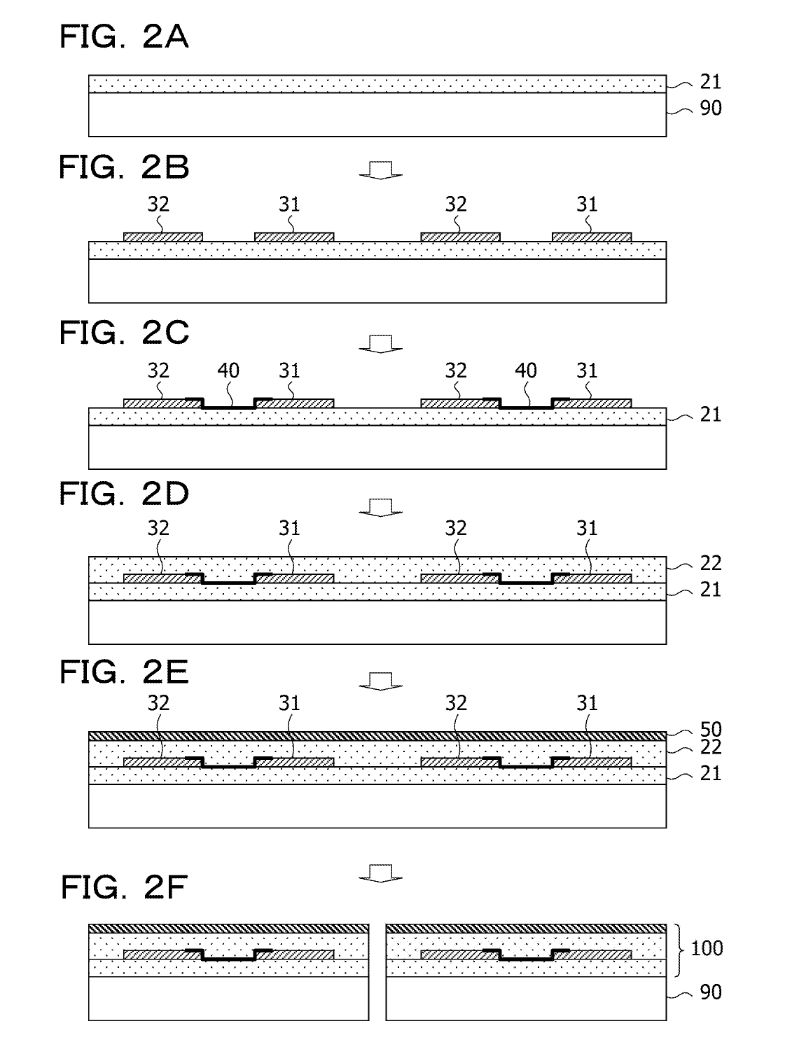

FIGS. 2A to 2F illustrate an example of a method of manufacturing the electronic device 100 illustrated in FIG. 1. The electronic device 100 is formed on a substrate 90 such as a silicone wafer or a resin. FIGS. 2A to 2F illustrate part of the substrate 90 on which two or more electronic devices 100 are formed.

First, polyethylene glycol having a molecular weight of 2500 is mixed with a solvent such as ethyl acetate, methyl ethyl ketone, or methylcyclohexane and heated to produce a mixed solution in which the solid content is dissolved. The mixed solution in which the solid content is dissolved is maintained in the liquid state even when the temperature is returned to room temperature. Then, the mixed solution having a predetermined viscosity is applied onto the substrate 90 such as a silicone wafer by spin-coating under room temperature. Then, the solvent is evaporated by drying, thereby the polymer film 21 is formed (FIG. 2A). The polymer film 21 is an example of a first polymer film. Here, the spin-coating is a technique in which a mixed solution of polyethylene glycol and a solvent is dropped onto the rotating substrate 90 so as to form a thin film by a centrifugal force. As described with reference to FIG. 1, the polymer film 21 may be formed of a meltable material or a material in which the meltable material is mixed with a non-meltable material as long as the meltable material (hereinafter referred to as “polymer material”) melts at a predetermined temperature higher than the body temperature.

The polymer film 21 may be formed by printing. In this case, a mask having openings is placed on the substrate 90. Each of the openings of the mask corresponds to the external shape of the electronic device 100. The meltable material or the mixture of the meltable material and the non-meltable material is melted by heating and then filled into the openings of the mask by using a squeegee. Thus, the polymer film 21 is formed. When a solvent is mixed with the meltable material or the mixture of the meltable material and the non-meltable material, the polymer film 21 is able to be printed at room temperature by using the mixed solution in which the solvent is mixed. In this case, ink jetting is able to be used.

Alternatively, the polymer film 21 may be formed by heating the meltable material or the mixture of the meltable material and the non-meltable material to the melting temperature and spraying the heated meltable material or the heated mixture of the meltable material and the non-meltable material onto the substrate 90. The polymer film 21 may be formed by spraying onto the substrate 90 a mixed solution in which the meltable material or the mixture of the meltable material and the non-meltable material is mixed with the solvent and then evaporating the solvent. In this case, the polymer film 21 is able to be formed at room temperature. Alternatively, the polymer film 21 formed in advance in the form of a film may be bonded onto the substrate 90 to form the polymer film 21.

Next, at, for example, room temperature, the electronic components 31, 32 and so forth are placed on the polymer film 21 by using a mounter (FIG. 2B). Next, the terminals of the electronic components 31 and 32 placed on the polymer film 21 are coupled to one another by the wiring 40 (FIG. 2C). For example, the wiring 40 is formed by applying by ink jetting Ag (silver) ink to a wiring region and drying.

When the wiring 40 is formed by ink jetting, the wiring 40 is able to be formed in a room temperature region. This may suppress melting of the polymer film 21. Further, when the melting temperature of the polymer film 21 is set to be higher than the melting temperature of the polymer film 22, for example, the temperature for drying the Ag ink is able to be increased compared to the case where the melting temperature of the polymer film 21 is the same as that of the polymer film 22. This may reduce the drying time. The wiring 40 may be formed of a conductive material other than the Ag ink. Alternatively, the wiring 40 may be formed by using a photolithography technique.

After the Ag ink has been dried, the polymer film 22 is formed on the polymer film 21 so as to cover the electronic components 31, 32 (FIG. 2D). The polymer film 22 is an example of a second polymer film. The polymer film 22 is formed by a technique similar to the technique used to form the polymer film 21. For example, the polymer film 22 is formed by spin-coating, printing, spraying, or film bonding.

However, in the case of forming the polymer film 22 by printing, printing is performed by squeezing or ink jetting at room temperature with a mixed solution in which a solvent is mixed with a meltable material or a mixed solution in which a solvent is mixed with the mixture of the meltable material and the non-meltable material. This may suppress a problem of melting of the polymer film 21 caused by adhesion of the material of the polymer film 22 at or higher than the melting temperature to the polymer film 21. In the case of spraying onto the polymer film 21 the polymer film 22 heated to a temperature at or higher than the melting temperature, the temperature of the material of the atomized polymer film 22 is reduced by the atmosphere, and the amount of the material of the polymer film 22 adhering to the polymer film 21 per unit time is small. Thus, the polymer film 21 is unlikely to melt even when the polymer film 22 adheres to the polymer film 21. In the case where the electronic component 32 is the display component and the display of the display component is exposed at the surface of the electronic device 100, the polymer film 22 is selectively formed in a region other than an upper surface of the electronic component 32 after the mask covering the upper surface of the electronic component 32 has been disposed.

After the polymer film 22 has been formed, for example, a mixed solution obtained by mixing a powdered silicone resin with a solvent such as ethyl acetate, methyl ethyl ketone, or methylcyclohexane is sprayed onto the polymer film 22. After that, when the solvent is evaporated, the hydrophobic film 50 providing hydrophobic performance is formed on the polymer film 22 (FIG. 2E). The hydrophobic film 50 may be formed by spraying a mixed solution obtained by mixing a powdered fluororesin with a solvent. Alternatively, the hydrophobic film 50 may be formed by spin-coating a mixed solution obtained by mixing a powdered silicone resin or a powdered fluororesin with a solvent or printing with a mixed solution obtained by mixing a powdered silicone resin or a powdered fluororesin with a solvent. In the case where the electronic component 32 is the display component and the display of the display component is exposed at the surface of the electronic device 100, the hydrophobic film 50 is selectively formed in a region other than an upper surface of the electronic component 32 after the mask covering the upper surface of the electronic component 32 has been disposed.

After a plurality of the electronic devices 100 are produced on the substrate 90 as described above, each electronic device 100 is cut out by dicing at the boundary of a device region of each electronic device 100 together with the substrate 90 (FIG. 2F). The electronic device 100 having been cut out is removed from the substrate 90 and wrapped so as not to be dried. At least one of the polymer films 21 and 22, the wiring 40, and the hydrophobic film 50 may be formed by three-dimensional (3D) printing.

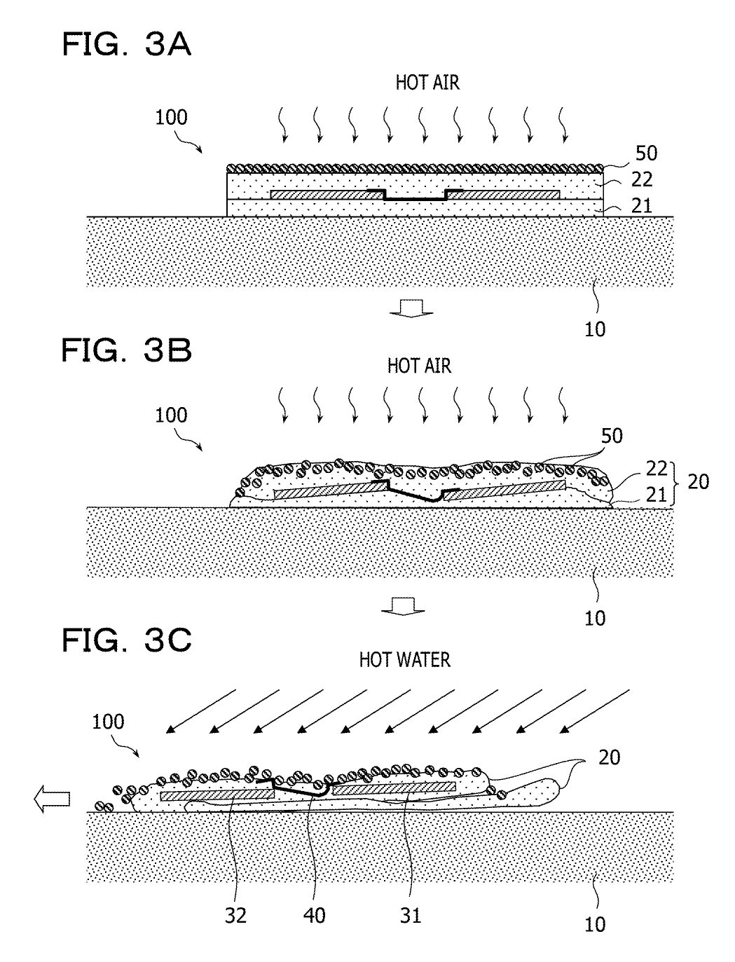

FIGS. 3A to 3C illustrate an example of a procedure for removing the electronic device 100 illustrated in FIG. 1 from the skin 10. For ease of description, the hydrophobic film 50 is illustrated in the form of particles. When removing the electronic device 100 from the skin 10, first, hot air is applied to the electronic device 100 by using a drier or the like (FIG. 3A). When the electronic device 100 is heated to a temperature at or higher than the melting temperature due to the hot air, the polymer films 21 and 22 gradually soften and start melting (FIG. 3B).

Here, in order to avoid burns, the heating temperature for the electronic device 100 is preferably about 60 to 70 degrees Celsius at the maximum. When the polymer films 21 and 22 melt, the function of the polymer film 22 as a base supporting for the hydrophobic film 50 is lost, thereby the hydrophobic film 50 is separated into pieces and incorporated into the polymer film 22. As a result, when part of the surface of the polymer film 22 is exposed from the hydrophobic film 50, the property of the surface of the electronic device 100 changes from hydrophobic to hydrophilic, and the hydrophobic function of the electronic device 100 provided by the hydrophobic film 50 is lost.

After the polymer film 20 has been deformed by melting and the hydrophobic film 50 has been incorporated into the polymer film 22, hot water is applied to the electronic device 100 by using a shower or the like. Part of the water-soluble polymer film 20 dissolves in the hot water and is washed off with hot water. At the same time, due to the pressure of the hot water, the polymer film 20 drops from the skin 10 together with the hydrophobic film 50 and the electronic components 31, 32 (FIG. 3C). Instead of the hot water, water may be applied to the electronic device 100. In the procedure illustrated in FIGS. 3A to 3C, there is no operation to pull the electronic device 100 from the skin 10. Thus, the electronic device 100 may be removed from the skin 10 without damaging the cuticles or the like of the skin 10.

In FIGS. 3A to 3C, hot air is applied to the entire surface of the electronic device 100 to melt the polymer film 20. Alternatively, the hot air may be locally applied to the electronic device 100 to locally melt the polymer film 20. In this case, part of the hydrophobic film 50 is incorporated into the locally melted polymer film 20, thereby openings that allow the hot water to be directly applied to the water-soluble polymer film 20 are formed. With the hot water entering through the openings, the polymer film 20 may be washed off from the skin 10 together with the hydrophobic film 50 and the electronic components 31, 32.

In the state illustrated in FIG. 3B, after the polymer film 20 has been deformed by melting and the hydrophobic film 50 has been incorporated into the polymer film 22, the melted electronic device 100 may be wiped off with a towel or the like so as to remove the electronic device 100 from the skin 10. The electronic components 31, 32, 33 (FIG. 1) removed from the skin 10 may be collected for reuse.

Thus, according to the embodiment illustrated in FIGS. 1 to 3C, the electronic device 100 attached to the skin 10 is able to be removed from the skin 10 by washing off from the skin 10 the polymer film 20 having been melted by heating together with the electronic components 30. In so doing, since the melted polymer film 20 has no adhesion, the electronic device 100 attached to the skin 10 may be removed from the skin 10 without damaging the skin 10.

With the hydrophobic film 50 formed on the surface of the polymer film 20, dissolution of the polymer film 20 is able to be suppressed even when water or the like is applied to the electronic device 100 attached to the skin 10. Thus, failure or the like of the electronic device 100 due to dissolution of the polymer film 20 may be suppressed. Since the hydrophobic film 50 formed on the polymer film 20 is incorporated into the polymer film 20 when the polymer film 20 melts, the hydrophobic function of the hydrophobic film 50 is able to be lost. Thus, the melted polymer film 20 together with the electronic components 30 may be washed off with hot water or the like. By setting the melting temperature of the polymer film 21 to be higher than the melting temperature of the polymer film 22, reaction between the polymer film 21 and sweat produced from the skin 10 may be suppressed.

FIG. 4 illustrates another embodiment of the electronic device. Elements that are the same as or similar to those described with reference to FIG. 1 are denoted by the same reference numerals, thereby omitting detailed description thereof. In an electronic device 100A illustrated in FIG. 4, an inclined portion 25 extending outward from the outer surface 24 toward the inner surface 23 is provided at the periphery of the polymer film 20 (21, 22). The hydrophobic film 50 is formed not only on the outer surface of the polymer film 20, but also on the inclined portion 25. Other structures of the electronic device 100A are similar to those of the electronic device 100 illustrated in FIG. 1.

With the inclined portion 25, sharpness of edge portions of the electronic device 100A is reduced. Thus, when an object strikes the electronic device 100A attached to the skin 10, shock applied to the electronic device 100A is reduced compared to the case where the of the electronic device 100 illustrated in FIG. 1 is used. This may reduce, compared to the case where the electronic device 100 is used, the frequency at which failure of the electronic device 100A due to the shock caused by being struck by the object occurs or problems with the electronic device 100A such as the removal of the electronic device 100A from the skin 10 and the like occur. Similarly to the description with reference to FIGS. 3A to 3C, the electronic device 100A drops from the skin 10 when the electronic device 100A is heated by hot air and then subjected to hot water or water.

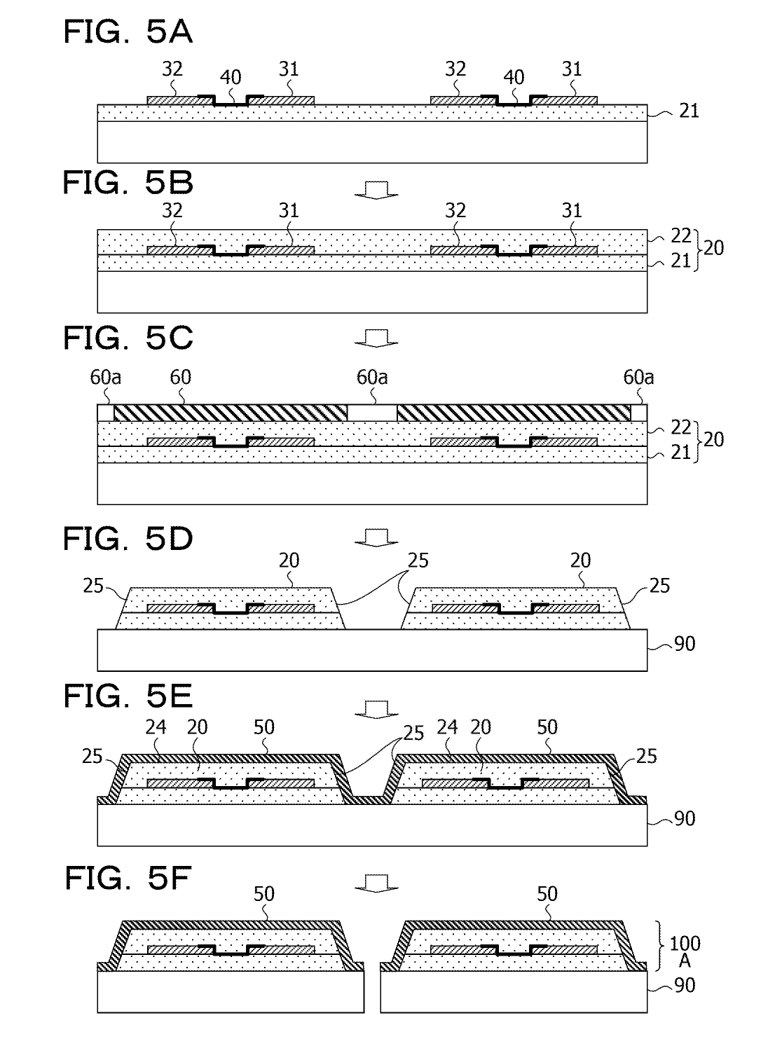

FIGS. 5A to 5F illustrate an example of a method of manufacturing the electronic device 100A illustrated in FIG. 4. Detailed description of steps that are the same as or similar to those illustrated in FIGS. 2A to 2F is omitted. First, steps illustrated in FIGS. 2A to 2C are performed. Thus, the terminals of the electronic components 31 and 32 placed on the polymer film 21 are coupled to one another by the wiring 40 (FIG. 5A). Next, similarly to the step illustrated in FIG. 2D, the polymer film 22 is formed on the polymer film 21 so as to cover the electronic components 31, 32 (FIG. 5B).

Next, a mask 60 having openings 60a corresponding to regions other than device regions of the electronic device 100A is placed on the polymer film 22 (FIG. 5C). Then, for example, free radicals generated by excitation of carbon tetrafluoride (CF₄) gas are reacted with the polymer film 20 exposed in each of the openings 60a by using isotropic dry etching. As a result, the polymer film 20 is isotropically etched in the substrate 90 direction and the device region direction from the opening 60a as a starting point. Thus, the inclined portion 25 is formed and the substrate 90 facing the openings 60a is exposed (FIG. 5D).

Next, similarly to the step illustrated in FIG. 2E, a solution obtained by mixing a silicone resin or a fluororesin with a solvent such as ethyl acetate, methyl ethyl ketone, or methylcyclohexane is sprayed onto the substrate 90. After that, the solvent is evaporated by drying. As a result, the hydrophobic film 50 is formed on the outer surface 24 and the inclined portion 25 of the polymer film 20 and the exposed portion of the substrate 90 (FIG. 5E). When the polymer film 20 has a stepped shape, the hydrophobic film 50 is desirably formed by spraying.

Since the inclined portion 25 is formed on the polymer film 20, when the hydrophobic film 50 is formed by spraying the solution onto the substrate 90, the hydrophobic film 50 having a predetermined thickness is able to be formed on the side walls (that is, the inclined portion 25) of the polymer film 20. Thus, the entire surface of the polymer film 20 is able to be covered with the hydrophobic film 50. Accordingly, the hydrophobic performance of the electronic device 100A to be attached to the skin 10 may be improved compared to that of the electronic device 100 illustrated in FIG. 1.

After that, similarly to the step illustrated in FIG. 2F, each electronic device 100 A is cut out by dicing the substrate 90 (FIG. 5F). In the electronic device 100A illustrated in FIG. 5F, the hydrophobic film 50 has a flange portion on the substrate 90 side. The flange portion is dropped when the electronic device 100A is removed from the substrate 90. The flange portion remaining in the electronic device 100A may be cut away. Alternatively, when the exposed portion of the substrate 90 is covered with a mask to form the hydrophobic film 50 after the step illustrated in FIG. 5D, the electronic device 100 A without the flange portion is able to be formed.

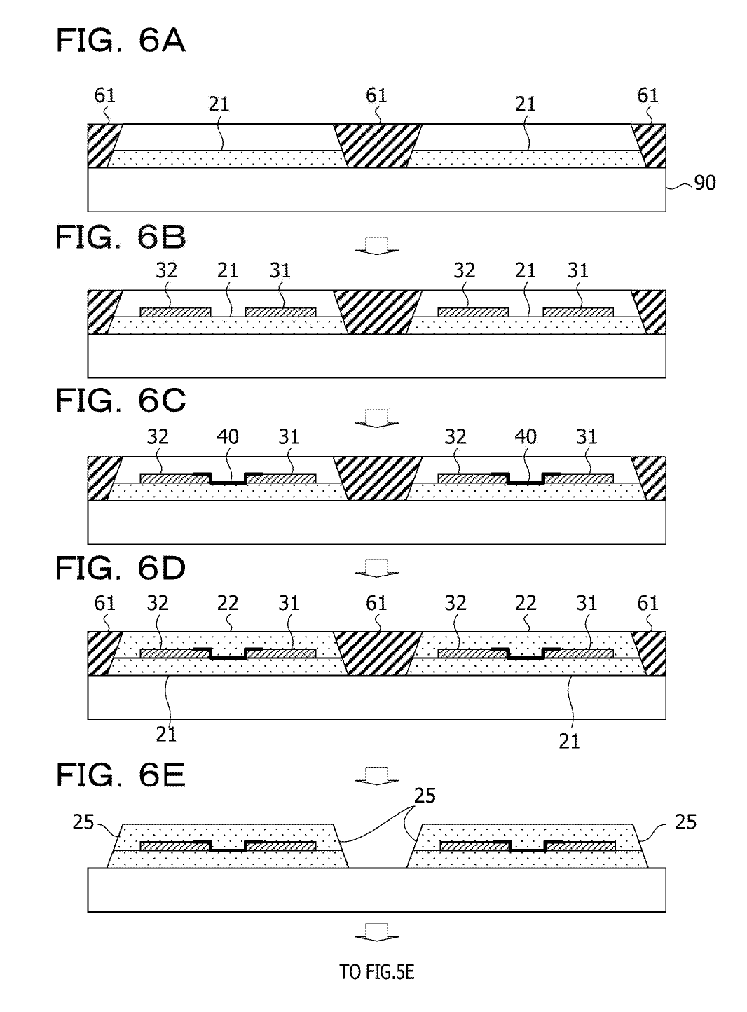

FIGS. 6A to 6E illustrate another example of the method of manufacturing the electronic device 100A illustrated in FIG. 4. Detailed description of the steps that are the same as or similar to those in FIGS. 2A to 2F or FIGS. 5A to 5F is omitted. In FIGS. 6A to 6E, before forming the polymer film 21, a mask 61 having inclined surfaces corresponding to the inclined portion 25 illustrated in FIG. 4 are placed on the substrate 90. Then, a mixture of a polymer material liquified due to heating and a solvent such as ethyl acetate, methyl ethyl ketone, or methylcyclohexane is sprayed onto the substrate 90, thereby the polymer film 21 is formed (FIG. 6A). When the polymer film 21 is formed by using the mask 61, the spin-coating, the printing, or the bonding of the film-shaped polymer film 21 described with reference to FIG. 2A is not used.

Next, similarly to the step illustrated in FIG. 2B, the electronic components 31, 32 are mounted on the polymer film 21 (FIG. 6B). Next, similarly to the step illustrated in FIG. 2C, the terminals of the electronic components 31 and 32 placed on the polymer film 21 are coupled to one another by the wiring 40 (FIG. 6C). Next, the polymer film 22 is formed on the polymer film 21 so as to cover the electronic components 31, 32 (FIG. 6D). Since the mask 61 is placed on the substrate 90, it is preferable that the polymer film 22 be formed by printing. The polymer film 22 may be formed by spraying.

Next, the mask 61 is removed from the substrate 90. As a result, the same structure as that of FIG. 5D remains on the substrate 90. After that, the steps described with reference to FIGS. 5E and 5F are sequentially performed, and the electronic device 100A illustrated in FIG. 4 is manufactured.

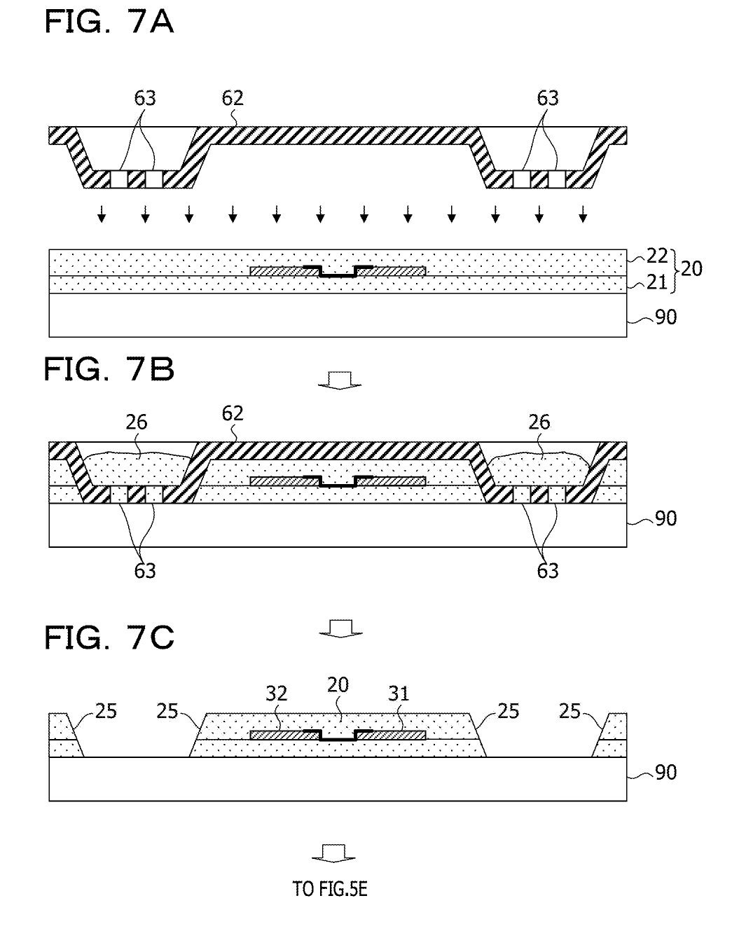

FIGS. 7A to 7C illustrate yet another example of the method of manufacturing the electronic device 100A illustrated in FIG. 4. Detailed description of the steps that are the same as or similar to those in FIGS. 2A to 2F or FIGS. 5A to 5F is omitted. Referring to FIGS. 7A to 7C, after the steps up to FIG. 5B have been performed, a plate-shaped mask 62 having inclined surfaces corresponding to the inclined portion 25 illustrated in FIG. 4 is placed on the polymer film 20 and pressed toward the substrate 90 (FIG. 7A). The mask 62 has through holes 63 at positions corresponding to the exposed portion of the substrate 90 in FIG. 7C.

When the mask 62 is pressed, waste matter 26 of the polymer film 20 existing at the position corresponding to the exposed portion of the substrate 90 flows out onto the mask 62 through the through holes 63 (FIG. 7B). After that, when the mask 62 is removed from the substrate 90, a structure similar to that in FIG. 5D remains (FIG. 7C). After that, the steps described with reference to FIGS. 5E and 5F are sequentially performed, and the electronic device 100A illustrated in FIG. 4 is manufactured.

When the mask 62 is pressed, a region of the polymer film 20 corresponding to the inclined portion 25 is deformed due to pressure. Thus, the distances between the electronic components 31, 32 and the inclined portion 25 are set to such distances that the deformation of the polymer film 20 does not affect a region where the electronic components 31, 32 are placed.

Thus, also according to the embodiment illustrated in FIGS. 4 to 7C, similarly to the embodiment illustrated in FIGS. 1 to 3C, when the electronic device 100A is heated, the electronic device 100A attached the skin 10 may be removed from the skin 10 without damaging the skin 10. Furthermore, according to the embodiment illustrated in FIGS. 4 to 7C, with the inclined portion 25, the entire surface of the polymer film 20 is able to be covered with the hydrophobic film 50. This may improve the hydrophobic performance of the electronic device 100A to be attached to the skin 10 compared to that of the electronic device 100 illustrated in FIG. 1. Furthermore, with the inclined portion 25, the frequency at which failure of the electronic device 100A occurs when an object strikes the electronic device 100A attached to the skin 10 may be reduced compared to the case where the electronic device 100 is used.

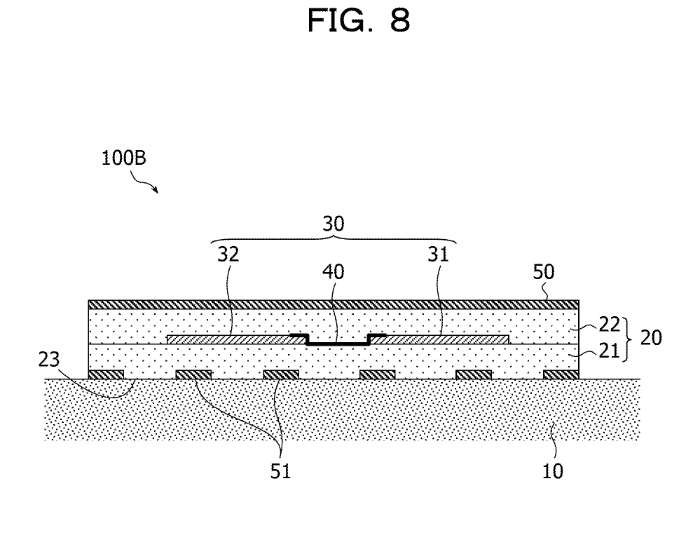

FIG. 8 illustrates another embodiment of the electronic device. Elements that are the same as or similar to those described with reference to FIG. 1 are denoted by the same reference numerals, thereby omitting detailed description thereof. An electronic device 100B illustrated in FIG. 8 is similar to the electronic device 100 illustrated in FIG. 1 except that, in the electronic device 100B, a hydrophobic film 51 is provided in partial regions of the inner surface 23 of the polymer film 21. The hydrophobic film 51 is an example of a second hydrophobic film. The hydrophobic film 51 is formed of a silicone resin or a fluororesin. The electronic device 100B is to be attached to the skin 10 utilizing the adhesive property of the polymer film 21 exposed from the hydrophobic film 51.

With the hydrophobic film 51 formed on parts of the surface of the electronic device 100 B to be in contact with the skin 10, dissolution of the entire inner surface 23 of the polymer film 21 due to sweat or the like may be suppressed while maintaining the adhesive property of the polymer film 21. For example, with the hydrophobic film 51, deformation of the electronic device 100B due to sweat may be suppressed.

When removing the electronic device 100B from the skin 10, first, the electronic device 100B is heated by hot air as described with reference to FIG. 3A. When heated, the polymer films 21 and 22 melt, and the silicone resin or the fluororesin of the hydrophobic films 50 and 51 is separated into pieces and incorporated into the polymer films 21 and 22. In this state, when hot water or water is applied to the electronic device 100B, the electronic device 100B drops from the skin 10.

A method of manufacturing the electronic device 100B is similar to the method of manufacturing illustrated in FIGS. 2A to 2F except that, in the method of manufacturing the electronic device 100B, the hydrophobic film 51 is selectively formed on the substrate 90 by using the mask before the polymer film 21 is formed on the substrate 90. As is the case with the hydrophobic film 50, the hydrophobic film 51 is formed by spraying onto the substrate 90 a solution obtained by mixing a silicone resin with a solvent such as ethyl acetate, methyl ethyl ketone, or methylcyclohexane. The hydrophobic film 51 may be formed of a fluororesin. The hydrophobic film 51 may be formed by, for example, printing, spin-coating, or 3D printing.

Thus, also according to the embodiment illustrated in FIG. 8, similarly to the embodiment illustrated in FIGS. 1 to 3C, when the electronic device 100B is heated, the electronic device 100B attached the skin 10 may be removed from the skin 10 without damaging the skin 10. Also according to the embodiment illustrated in FIG. 8, the hydrophobic film 51 formed on the inner surface 23 side may suppress deformation of the electronic device 100B caused by dissolution of the polymer film 21 due to sweat or the like while maintaining the adhesive property of the polymer film 21.

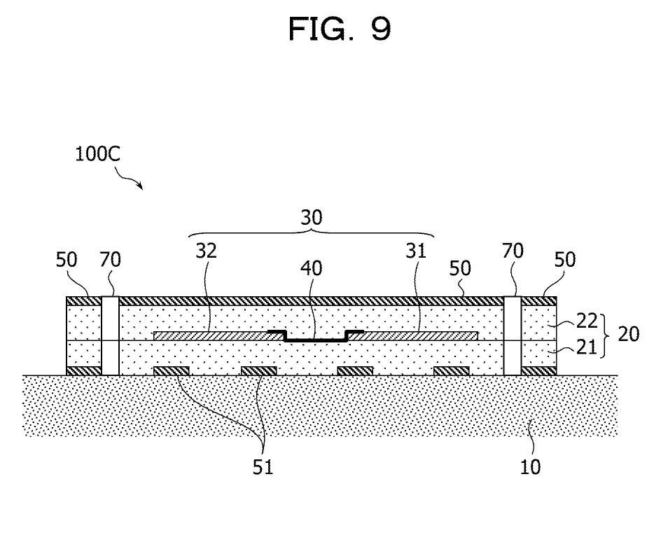

FIG. 9 illustrates another embodiment of the electronic device. Elements that are the same as or similar to those described with reference to FIGS. 1 and 8 are denoted by the same reference numerals, thereby omitting detailed description thereof. An electronic device 100C illustrated in FIG. 9 has a structure similar to that of the electronic device 100B illustrated in FIG. 8 except that the electronic device 100C has vents 70 extending through a range from the hydrophobic film 50 to the hydrophobic film 51.

The vents 70 may allow part of the sweat (water vapor) produced from the skin 10 covered with the electronic device 100C to be released. Thus, the amount of dissolution of the polymer film 21 due to sweat may be reduced compare to that of the polymer film 21 of the electronic device 100B illustrated in FIG. 8. Similarly to the description with reference to FIGS. 3A to 3C and 8, the electronic device 100C drops from the skin 10 when the electronic device 100C is heated by hot air and then subjected to hot water or water.

The electronic device 100C is manufactured by forming the vents 70 extending through to the substrate 90 by, for example, laser processing or etching after the electronic device 100B illustrated in FIG. 8 has been formed on the substrate 90. The method of manufacturing the electronic device 100C is similar to the method of manufacturing illustrated in FIG. 8 except that, in the method of manufacturing the electronic device 100C, the vents 70 are formed. The method of manufacturing the electronic device 100C is similar to the method of manufacturing illustrated in FIGS. 2A to 2F except that, in the method of manufacturing the electronic device 100C, the hydrophobic film 51 and the vents 70 are formed. The vents 70 may be formed in the electronic device 100 illustrated in FIG. 1 or the electronic device 100A illustrated in FIG. 4.

Thus, also according to the embodiment illustrated in FIG. 9, similarly to the embodiment illustrated in FIGS. 1 to 8, when the electronic device 100C is heated, the electronic device 100C attached the skin 10 may be removed from the skin 10 without damaging the skin 10. Furthermore, the hydrophobic film 51 may suppress deformation of the electronic device 100C caused by dissolution of the polymer film 21 due to sweat or the like while maintaining the adhesive property of the polymer film 21.

Furthermore, according to the embodiment illustrated in FIG. 9, the vents 70 may allow part of the sweat (water vapor) produced from the skin 10 covered with the electronic device 100C to be released. Thus, the amount of dissolution of the polymer film 21 due to sweat may be reduced compare to that of the polymer film 21 of the electronic device 100B illustrated in FIG. 8.

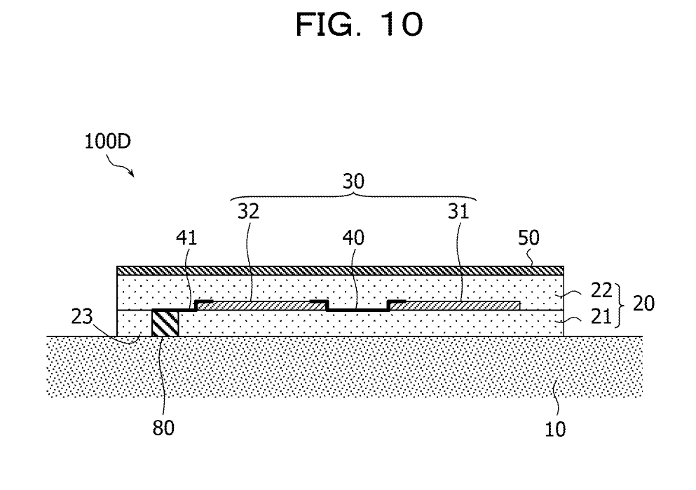

FIG. 10 illustrates another embodiment of the electronic device. Elements that are the same as or similar to those described with reference to FIG. 1 are denoted by the same reference numerals, thereby omitting detailed description thereof. An electronic device 100D illustrated in FIG. 10 has a structure similar to that of the electronic device 100 illustrated in FIG. 1 except that the electronic device 100D includes an electrode 80 to be brought into contact with the skin 10. The electrode 80 is coupled to the terminal of the electronic component 32 through wiring 41. As a result, the electronic component 32 is able to directly detect the biological information from the skin 10 through the electrode 80. For example, when acquiring an electrocardiogram of a patient or the like, an ammeter is used as the electronic component 32, and the electrode 80 is used as a current measuring terminal coupled to the ammeter.

In addition, the electrode 80 is formed not in the entirety of the inner surface 23 of the polymer film 22. Thus, irritation to the skin 10 by the electrode 80 may be minimized and the adhesive property of the polymer film 21 to the skin 10 is able to be maintained. Similarly to the description with reference to FIGS. 3A to 3C, the electronic device 100D drops from the skin 10 when the electronic device 100D is heated by hot air and then subjected to hot water or water.

The vents 70 illustrated in FIG. 9 may be formed in the electronic device 100D. Furthermore, the electrode 80 may be formed in the electronic device 100A illustrated in FIG. 4. Furthermore, the vents 70 illustrated in FIG. 9 and the electrode 80 may be formed in the electronic device 100A illustrated in FIG. 4.

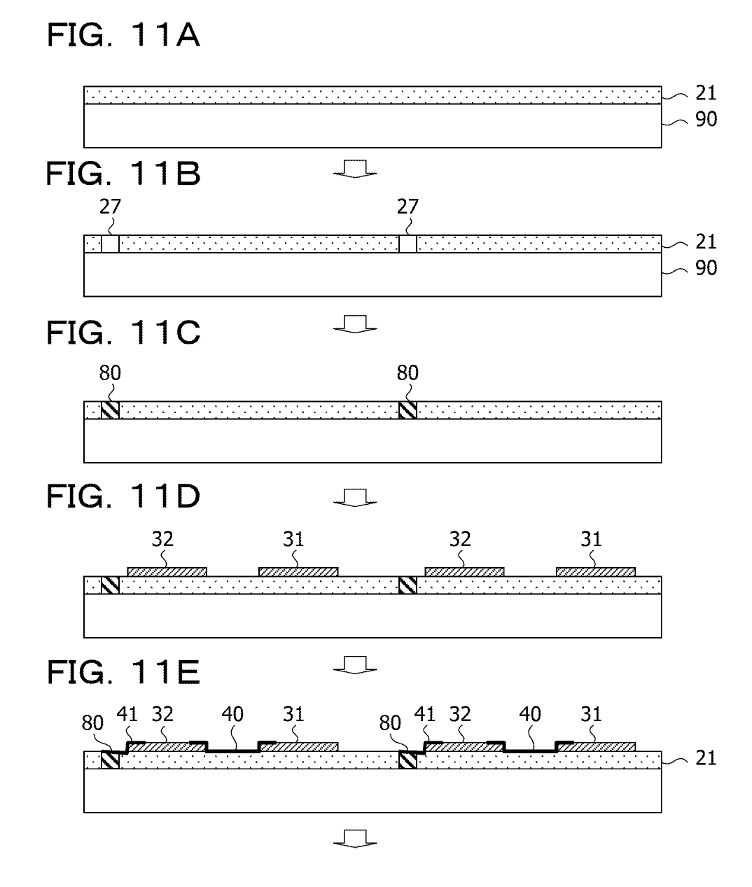

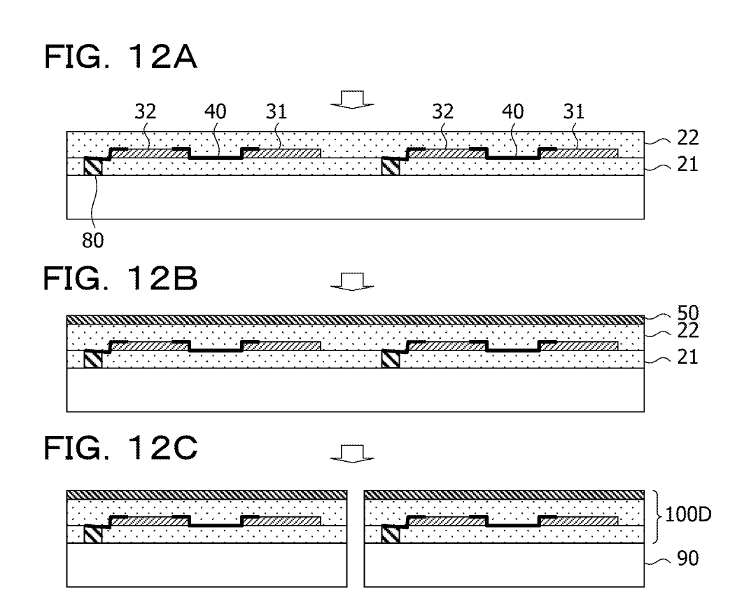

FIGS. 11A to 12C illustrates an example of a method of manufacturing the electronic device 100D illustrated in FIG. 10. Detailed description of steps that are the same as or similar to those illustrated in FIGS. 2A to 2F is omitted. First, similarly to the step illustrated in FIG. 2A, the polymer film 21 is formed on the substrate 90 by spin-coating, printing, spraying, or film bonding (FIG. 11A). Next, a through hole 27 extending through the polymer film 21 to the substrate 90 is formed by, for example, laser beam machining or etching (FIG. 11B). Next, the through hole 27 is filled with Ag ink or the like by ink jetting, thereby the electrode 80 is formed in the polymer film 21 (FIG. 11C).

Next, similarly to the step illustrated in FIG. 2B, the electronic components 31, 32 are placed on the polymer film 21 (FIG. 11D). Next, similarly to the step illustrated in FIG. 2C, the terminals of the electronic components 31 and 32 placed on the polymer film 21 are coupled to one another by the wiring 40. Furthermore, a terminal of the electronic component 32 and another end of the electrode 80 are coupled to each other by the wiring 41 (FIG. 11E). Alternatively, the terminal of the electronic component 32 may be directly coupled to the electrode 80 by placing the electronic component 32 on the polymer film 21 so as to cover the electrode 80. In this case, the wiring 41 is not formed.

Next, similarly to the step illustrated in FIG. 2D, the polymer film 22 is formed on the polymer film 21 so as to cover the electronic components 31, 32 and the electrode 80 (FIG. 12A). Next, similarly to the step illustrated in FIG. 2E, the hydrophobic film 50 containing a silicone resin or a fluororesin is formed on the polymer film 22 (FIG. 12B). After that, similarly to the step illustrated in FIG. 2F, each electronic device 100D is cut out by dicing the substrate 90. The electronic device 100D having been cut out is removed from the substrate 90, thereby the electronic device 100D is completed. At least one of the polymer film 21, the polymer film 22, the electrode 80, the wiring 40, the wiring 41, and the hydrophobic film 50 may be formed by 3D printing.

Thus, also according to the embodiment illustrated in FIGS. 10 to 12C, similarly to the embodiment illustrated in FIGS. 1 to 3C, when the electronic device 100D is heated, the electronic device 100D attached the skin 10 may be removed from the skin 10 without damaging the skin 10. Furthermore, according to the embodiment illustrated in FIGS. 10 to 12C, the skin 10 and the electronic component 32 are directly electrically coupled to each other through the electrode 80. This allows the electronic component 32 to detect the biological information directly from the skin 10 through the electrode 80. Accordingly, compared to the case where the biological information is detected through the polymer film 21, the sensitivity for detecting the biological information may be improved. In other words, the electronic device 100D is able to detect such biological information that is difficult to detect with the electronic device 100 illustrated in FIG. 1. Furthermore, the electrode 80 is formed not in the entirety of the inner surface of the polymer film 22. Thus, irritation to the skin 10 by the electrode 80 may be minimized and the adhesive property of the polymer film 21 to the skin 10 is able to be maintained.

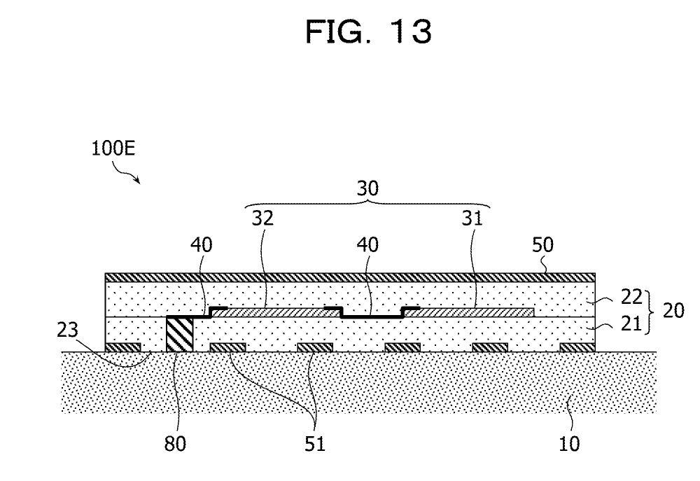

FIG. 13 illustrates another embodiment of the electronic device. Elements that are the same as or similar to those described with reference to FIGS. 1, 8, and 10 are denoted by the same reference numerals, thereby omitting detailed description thereof. An electronic device 100E illustrated in FIG. 13 is similar to the electronic device 100D illustrated in FIG. 10 except that, in the electronic device 100E, a hydrophobic film 51 is provided in partial regions of the inner surface 23 of the polymer film 20. Similarly to the description with reference to FIGS. 3A to 3C and 8, the electronic device 100E drops from the skin 10 when the electronic device 100E is heated by hot air and then subjected to hot water or water.

A method of manufacturing the electronic device 100E is similar to the method of manufacturing illustrated in FIGS. 11A to 12C except that, in the method of manufacturing the electronic device 100E, the hydrophobic film 51 is formed on the substrate 90 similarly to the description with reference to FIG. 8 before the polymer film 21 is formed on the substrate 90. The vents 70 illustrated in FIG. 9 may be formed in the electronic device 100E. Thus, also according to the embodiment illustrated in FIG. 13, the effects similar to those obtain according to the embodiments illustrated in FIGS. 1 to 3C, FIG. 8, and FIGS. 10 to 12C may be obtained.

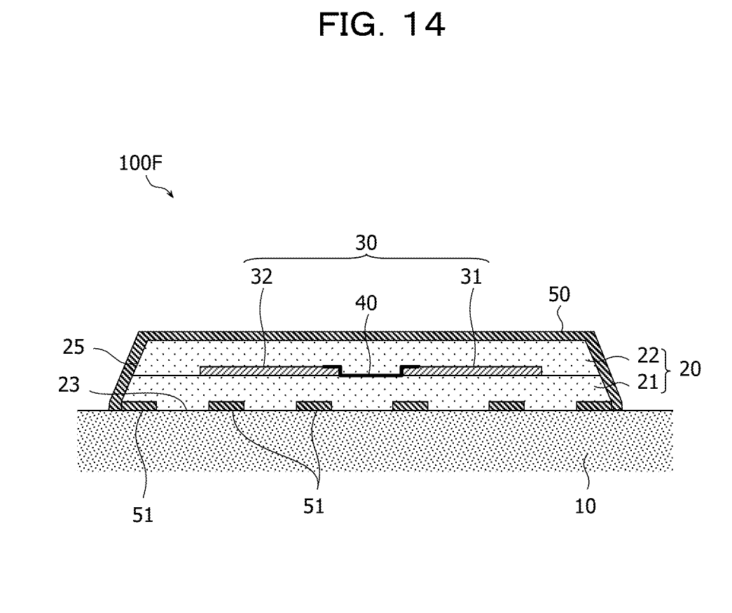

FIG. 14 illustrates another embodiment of the electronic device. Elements that are the same as or similar to those described with reference to FIGS. 1, 4, and 8 are denoted by the same reference numerals, thereby omitting detailed description thereof. An electronic device 100F illustrated in FIG. 14 is similar to the electronic device 100A illustrated in FIG. 4 except that, in the electronic device 100F, a hydrophobic film 51 is provided in partial regions of the inner surface 23 of the polymer film 20. The vents 70 illustrated in FIG. 9 may be formed in the electronic device 100F, the electrode 80 illustrated in FIG. 10 may be formed in the electronic device 100F, or the vents 70 illustrated in FIG. 9 and the electrode 80 illustrated in FIG. 10 may be formed in the electronic device 100F. Similarly to the description with reference to FIGS. 3A to 3C and 8, the electronic device 100F drops from the skin 10 when the electronic device 100F is heated by hot air and then subjected to hot water or water.

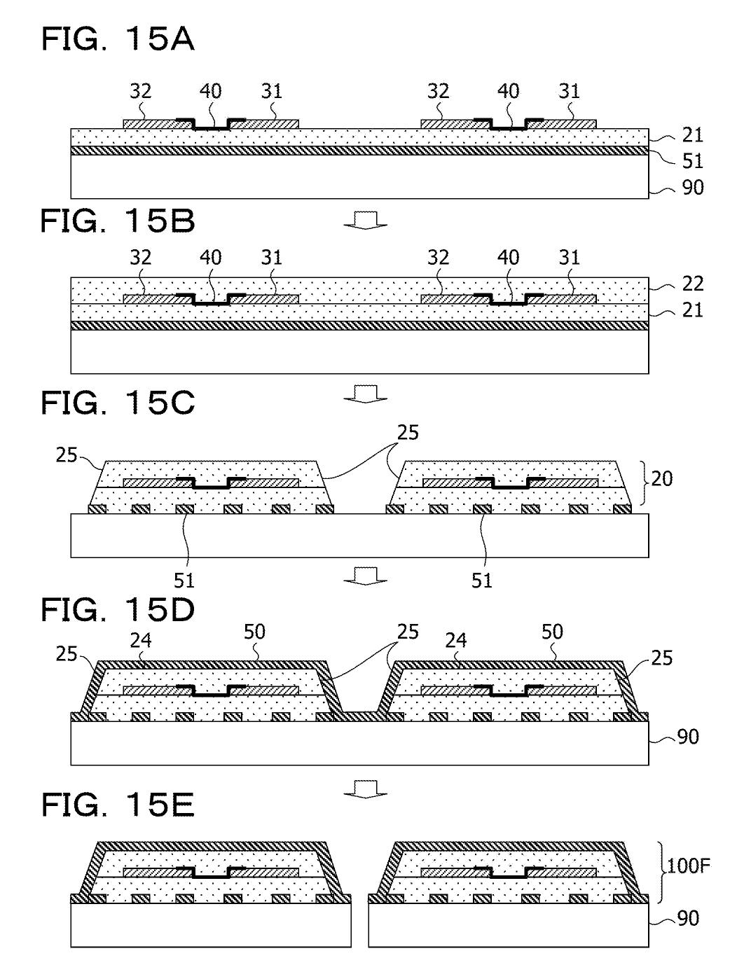

FIGS. 15A to 15E illustrate an example of a method of manufacturing the electronic device 100F illustrated in FIG. 14. Detailed description of the steps that are the same as or similar to those in FIGS. 2A to 2F and FIGS. 5A to 7C are omitted. A method of manufacturing the electronic device 100F is similar to the method of manufacturing illustrated in FIGS. 5A to 5F except that, in the method of manufacturing the electronic device 100F, the hydrophobic film 51 is formed on the substrate 90 before the polymer film 21 is formed on the substrate 90.

First, after the hydrophobic film 51 has been formed on the substrate 90, steps similar to the steps illustrated in FIGS. 2A to 2C are performed. Thus, the terminals of the electronic components 31 and 32 placed on the polymer film 21 are coupled to one another by the wiring 40 (FIG. 15A). Next, similarly to the step illustrated in FIG. 2D, the polymer film 22 is formed on the polymer film 21 so as to cover the electronic components 31, 32 (FIG. 15B).

Next, similarly to the steps in FIG. 5C and FIG. 5D, the inclined portion 25 is formed around the polymer film 20 by isotropic dry etching (FIG. 15C). In so doing, the hydrophobic film 51 exposed in the opening portion between the inclined portions 25 is removed by dry etching. Next, similarly to the step illustrated in FIG. 5E, the hydrophobic film 50 is formed on the outer surface 24 and the inclined portion 25 of the polymer film 20 and the exposed portion of the substrate 90 (FIG. 15D). After that, similarly to the step illustrated in FIG. 5F, each electronic device 100F is cut out by dicing the substrate 90 (FIG. 15E).

When the electronic device 100F is manufactured by utilizing the mask 61 as illustrated in FIGS. 6A to 6E, the mask 61 may be placed on the substrate 90 before the hydrophobic film 51 is formed or placed on the hydrophobic film 51 after the hydrophobic film 51 has been formed. When the hydrophobic film 51 is formed after the mask 61 is placed on the substrate 90, the hydrophobic film 51 is formed by spraying. When the mask 61 is placed on the hydrophobic film 51 after the hydrophobic film 51 is formed, the hydrophobic film 51 is formed by spraying, printing, or spin-coating. When the electronic device 100F is manufactured by utilizing the mask 62 as illustrated in FIGS. 7A to 7C, instead of the step described with reference to FIG. 15C, the steps illustrated in FIGS. 7A to 7C is performed. Thus, also according to the embodiment illustrated in FIGS. 14 to 15E, the effects similar to those obtain according to the embodiments illustrated in FIGS. 1 to 3C and FIGS. 4 to 8 may be obtained.

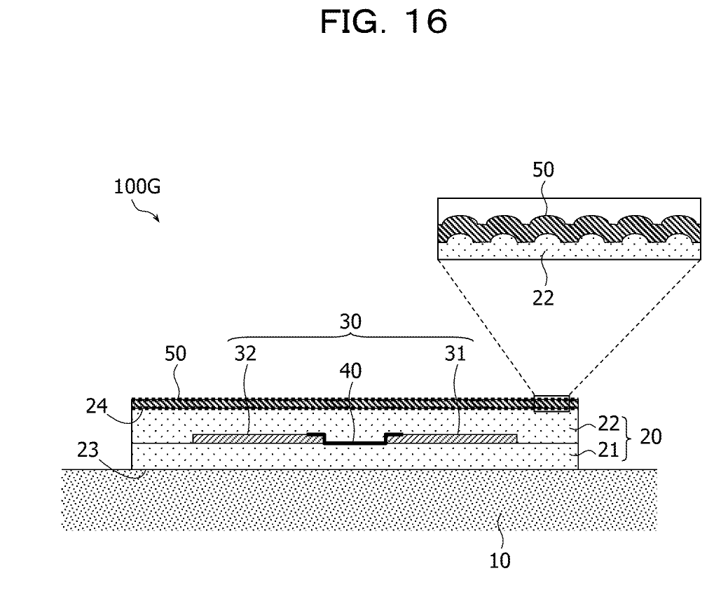

FIG. 16 illustrates another embodiment of the electronic device. Elements that are the same as or similar to those described with reference to FIG. 1 are denoted by the same reference numerals, thereby omitting detailed description thereof. An electronic device 100G illustrated in FIG. 16 has a structure similar to that of the electronic device 100 illustrated in FIG. 1 except that, in the electronic device 100G, the outer surface 24 of the polymer film 22 and the surface of the hydrophobic film 50 have uneven patterns. With the uneven pattern formed on the surface of the hydrophobic film 50, the hydrophobic function may be improved compared to the case where the hydrophobic film 50 is flat. Similarly to the description with reference to FIGS. 3A to 3C, the electronic device 100G drops from the skin 10 when the electronic device 100G is heated by hot air and then subjected to hot water or water.

A method of manufacturing the electronic device 100G is similar to the method illustrated in FIGS. 2A to 2F except that the method of manufacturing the electronic device 100G further includes a step of embossing between the steps illustrated in FIGS. 2D and 2E. The embossed uneven pattern of the polymer film 22 is formed by pressing the polymer film 22 with a die having an irregular pattern engraved thereon so as to deform the surface of the polymer film 22 after the step illustrated in FIG. 2D has been performed. After that, similarly to the step illustrated in FIG. 2E, when a solution obtained by mixing a silicone resin or a fluororesin with a solvent is sprayed onto the polymer film 22, the hydrophobic film 50 having the uneven pattern corresponding to the uneven pattern of the polymer film 22 is formed.

The uneven pattern may be formed only on the surface of the hydrophobic film 50 by pressing the die on which the uneven pattern is engraved against the hydrophobic film 50 after the hydrophobic film 50 has been formed on the polymer film 22 by performing the steps illustrated in FIGS. 2A to 2E. The uneven pattern may be formed only on the surface of the hydrophobic film 50 by laser beam machining, etching, or the like after the hydrophobic film 50 has been formed on the polymer film 22 by performing the steps illustrated in FIGS. 2A to 2E. The hydrophobic film 50 having the uneven pattern may be formed by 3D printing after the polymer film 22 has been formed by performing the steps illustrated in FIGS. 2A to 2D. When the hydrophobic function is realized by the uneven pattern, the hydrophobic film 50 may be formed of a material other than a silicone resin or a fluororesin.

Similarly to the electronic device 100A illustrated in FIG. 4, the electronic device 100G may have the inclined portion 25. In this case, the uneven pattern is formed on a flat portion of the hydrophobic film 50 other than the inclined portion 25. Similarly to the electronic device 100 B illustrated in FIG. 8, the electronic device 100G may have the hydrophobic film 51. Similarly to the electronic device 100C illustrated in FIG. 9, the electronic device 100G may have the vents 70. Similarly to the electronic device 100D illustrated in FIG. 10, the electronic device 100G may include the electrode 80. The electronic device 100G may include at least two types of the elements including the inclined portion 25, the hydrophobic film 51, the vents 70, and the electrode 80.

Thus, also according to the embodiment illustrated in FIG. 16, the effects similar to those obtain according to the embodiment illustrated in FIGS. 1 to 3C may be obtained. Furthermore, according to the embodiment illustrated in FIG. 16, the uneven pattern is formed on the surface of the hydrophobic film 50. Thus, hydrophobic function may be improved compared to the case where the hydrophobic film 50 is flat.

With the detailed description having been described, the features and advantages of the embodiments will become apparent. This is intended to extend to the features and advantages of the embodiments as described above as long as the claims are not departing from the gist of the claims and the scope of right. Also, one skilled in the art is able to easily made any modifications and changes. Accordingly, it is not intended to limit the scope of the patentable embodiments to the above description. The patentable embodiments are also able to be based on appropriate improvement or equivalents included in the scope of the disclosure in the embodiments.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. An electronic device comprising:
a polymer film that is to melt at a predetermined temperature higher than a body temperature;
at least one electronic component provided in the polymer film; and
a first hydrophobic film provided on an opposite surface of the polymer film to a side of the polymer film to be attached to skin.

2. The electronic device according to claim 1, wherein
an inclined portion inclined outward from the opposite surface toward the side to be attached to the skin is provided in a periphery of the polymer film, and
wherein the first hydrophobic film has a shape covering the opposite surface and the inclined portion.

3. The electronic device according to claim 1, further comprising:
a second hydrophobic film provided on the side of the polymer film to be attached to the skin.

4. The electronic device according to claim 3, wherein
the second hydrophobic film includes a silicone resin or a fluororesin.

5. The electronic device according to claim 1, further comprising:
a vent extending through from a surface of the first hydrophobic film to the side of the polymer film to be attached to the skin.

6. The electronic device according to claim 1, further comprising:
an electrode provided in the polymer film so as to be exposed to the side to be attached to the skin.

7. The electronic device according to claim 1, wherein
the polymer film includes
a first polymer film provided on the side to be attached to the skin, and
a second polymer film provided on an opposite side to the side to be attached to the skin,
wherein the at least one electronic component is disposed at an interface between the first polymer film and the second polymer film, and
wherein a melting temperature of the first polymer film is higher than a melting temperature of the second polymer film.

8. The electronic device according to claim 1, wherein
the polymer film includes at least one of polyethylene glycol and gelatin.

9. The electronic device according to claim 8, wherein
the polymer film further includes at least one of collagen and starch.

10. The electronic device according to claim 1, wherein
the first hydrophobic film includes a silicone resin or a fluororesin.

11. The electronic device according to claim 1, wherein
a surface of the first hydrophobic film has an uneven pattern.

12. A method of manufacturing an electronic device, the method comprising:
forming over a substrate a first polymer film that is to melt at a predetermined temperature higher than a body temperature;
placing at least one electronic component over the first polymer film;
forming a second polymer film that is to melt at a predetermined temperature higher than the body temperature on the first polymer film so as to cover the at least one electronic component;
forming a first hydrophobic film over the second polymer film; and
removing from the substrate a device region of the electronic device including the first polymer film, the at least one electronic component, the second polymer film, and the first hydrophobic film.

13. The method according to claim 12, further comprising:
removing the second polymer film and the first polymer film from a periphery of the device region after the forming of the second polymer film so as to form at the periphery of the device region an inclined portion inclined outward from a second polymer film side to a first polymer film side,
wherein the first hydrophobic film is formed so as to cover a surface of the second polymer film and the inclined portion.

14. The method according to claim 12, further comprising:
forming a second hydrophobic film over the substrate before the forming of the first polymer film over the substrate,
wherein the first polymer film is formed over the substrate with the second hydrophobic film interposed therebetween.

15. The method according to claim 12, further comprising:
forming a vent extending through from the first hydrophobic film to the substrate at a time after the forming of the first hydrophobic film over the second polymer film and before the removing of the device region from the substrate.

16. The method according to claim 12, further comprising:
forming a through hole extending through the first polymer film to the substrate at a time after the forming of the first polymer film over the substrate and before the placing of the at least one electronic component over the first polymer film; and
forming in the through hole an electrode to be coupled to the at least one electronic component.

17. The method according to claim 12, wherein
the at least one electronic component includes a plurality of the electronic components, and
wherein the method further includes connecting the plurality of the electronic components which are placed on the first polymer film to one another through wiring before the forming of the second polymer film.
